(19) United States
(12) Patent Application Publication
(10) Pub. No.: US 2007/0066966 A1
(43) Pub. Date: Mar. 22, 2007

(54) Implantable medical device

(76) Inventor: Christopher Davey, Boston, MA (US)

Correspondence Address: Kevin M. Farrell;Pierce Atwood, Suite 350, One New Hampshire Avenue, Portsmouth, NH, 03801, US

(21) Appl. No.: 11/242,101

(22) Filed: Oct. 3, 2005

(51) Int. Cl. A61K 9/22

(52) U.S. Cl. 604/891.1

(57) ABSTRACT

The device of the present invention includes a modular, implantable catheterization port that is composed of a body portion, an interior port and an exterior port. The interior and exterior ports are selectively attachable to the body portion such that the device can be assembled by a clinician at the skin entry site without compromising the position of other portions of the device within and outside of the patient. The device further includes a first skirt and a second skirt that surround the exterior port at its junction with the body portion. Both skirts are designed to gradually affix themselves into the surrounding tissues of the patient's body during the initial phase of healing. The first skirt is composed of a bio-absorbable material such that in subsequent phases of healing its connection to the surrounding tissues diminishes, leaving the second skirt to maintain a mature, secure, and permanent connection to the tissues. The bio-absorbable material can be selected prior to assembly in order to ensure that the first skirt is fully absorbed at the conclusion of the procedure, thus permitting an easy and less-invasive removal of the device from the patient.

### BACKGROUND OF THE PRESENT INVENTION

1. Field of the Invention

The present invention relates generally to the field of medical devices and in particular to the field of long term, implantable devices for permitting access to a patient's inner physiology.

2. Summary of the Related Art

It is often necessary in medically treating a patient to establish long term vascular access to a specific desired interior body site for purposes of administering liquid therapeutic agents, for removing bodily fluids for testing or monitoring, for treatment of bodily fluids before being returned to the body, or for disposal of bodily fluids. In another increasingly common medical procedure, it is desired to deliver a contained heat exchange fluid at a temperature above or below normal body temperature to a specific interior body site for providing localized or general heating or cooling. In still other common medical procedures, such as angioplasty and laparoscopy, medical instrumentation is guided through a pre-positioned catheter line to a particular internal body location to monitor body conditions and/or to perform medical/surgical procedures.

Particularly in the case of administering fluids to, or removing fluids from, the body continuously or periodically over an extended time period, it is known in the medical arts to use what are known as “permanent” catheterization techniques employing implanted devices such as tunneled central venous catheters (CVCs) for durations ranging from a few weeks to years. Examples of such implanted and related medical devices are found in U.S. Pat. No. 4,266,999 (Baier); U.S. Pat. No. 4,405,305 (Stephen et al.); U.S. Pat. No. 4,488,877 (Klein et al.); U.S. Pat. No. 4,668,222 (Poirier); U.S. Pat. No. 4,897,081 (Poirier et al.); U.S. Pat. No. 4,935,004 (Cruz); U.S. Pat. No. 5,098,397 (Svensson et al.); U.S. Pat. No. 5,100,392 (Orth et al.); U.S. Pat. No. 5,242,415 (Kantrowitz et al.); U.S. Pat. No. 5,662,616 (Bousquet); U.S. Pat. No. 5,823,994 (Sharkey et al.); U.S. Pat. No. 5,830,184 (Basta); U.S. Pat. No. 5,848,987 (Baudino et al.); U.S. Pat. No. 5,882,341 (Bousquet); U.S. Pat. No. 5,989,213 (Maginot); and U.S. Pat. No. 6,033,382 (Basta), each of which is incorporated herein by reference. Examples of therapeutic regimens requiring such long-term continuous or periodic access to a specific internal body location include parenteral feeding, chemotherapy, antibiotic administration, dialysis, and others.

Generally, the length of time the patient will be catheterized dictates whether a physician will utilize a “temporary” catheterization technique or a “permanent” catheterization technique For example, a procedure in which a clot is aspirated from a blood vessel typically includes placing the catheter in the blood vessel for a relatively short period of time such as a few minutes to a few hours and then withdrawing the catheter once the clot has been removed. Therefore, when performing such an aspiration procedure, it is common for a physician to use the temporary catheterization technique to place the catheter in the blood vessel of the patient.

On the other hand, when a procedure is performed to effect hemodialysis, a physician may place a catheter in the blood vessel for a relatively long period of time. In particular, a patient suffering from kidney failure who is involved in a hemodialysis regimen typically requires a dialysis session three times per week for an indefinite period of time whereby extra fluid, chemicals, and wastes are removed from her body. A patient who is involved in such a hemodialysis regimen may need a catheter placed in her blood vessel for a relatively long period of time in order to provide a ready means for vascular access into his/her bloodstream over such relatively long period of time.

Permanent catheterization techniques typically entail inserting a “permanent” catheter into a patient's blood vessel using a “tunneled catheter technique.” Although done regularly, current CVC designs seriously compromise the skin's ability to protect the body from infection. CVC-related infection is a serious health problem that significantly increases the morbidity rate and cost of catheter usage. All previous attempts to modify tunneled CVC designs to reduce infection have failed to significantly decrease this cost or the morbidity rate. The primary reason for the failure of conventional tunneled CVCs is that none of the modified versions effectively block the most significant path of microorganisms through the skin and into the body. Although tunneled CVC's typically include a tissue in-growth cuff that is intended to anchor the device in the sub-cutaneous tunnel and establish a barrier for micro-organisms entering the body, these conventional devices still produce undesirably high infection rates because the cuff cannot function in a manner that can establish this barrier in the most effective location, namely at the skin entry site. Additionally, conventional cuffs are secured to the device in a fixed location during manufacture, which imposes limits on the ability of the clinician to optimize the position of the cuff relative to the patient's unique anatomy.

Unlike other types of implanted devices, such as peritoneal dialysis catheters, tunneled CVCs need to have their tips placed in a very specific location, typically the Superior Vena Cava/Right Atrial Junction (SVC/RA), in order to function properly over many months. Interventional radiologists are acutely aware of the need for highly precise tip placement because they are most frequently called on to resolve CVC complications. With currently available technology, the ability to precisely position CVC tips in the SVC/RA is enabled largely by the freedom to adjust/position the cuff location anywhere within the subcutaneous tunnel length. Thus, conventional devices do not allow clinicians to concurrently optimize the placement of the cuff and the tip in precise locations.

It has now been found, however, that a CVC apparatus which includes an adjustable epidermal tissue ingrowth cuff assembly according to the present invention overcomes these problems and deficiencies of the prior art CVC devices. For example, the apparatus and methods disclosed in U.S. Patent Application No. 2004/0236314 to Mark A. Saab, incorporated herein by reference, allow a physician to place a fixed epidermal tissue ingrowth cuff assembly within a skin incision site and, subsequently, to adjust the location of the distal (internal) tip of a catheter assembly associated with the tissue ingrowth cuff assembly to precisely position the catheter tip at the desired body site without disturbing, moving, or stressing the fixed tissue ingrowth cuff.

Nevertheless, while development of advanced tissue ingrowth cuff assemblies have resulted in numerous improvements related to patient care and health, a typical epidermal tissue ingrowth cuff has a significantly larger surface area than conventional cuffs, resulting in significantly more tissue ingrowth, which can lead to difficulty in removing the device when it is no longer needed or needs to be replaced. There is a need to provide an adequately large surface area cuff that promotes stable and secure attachment to the skin tissue during the tenuous early stages of the healing, but does not provide such an excessively robust tissue ingrowth that once the healing process is completed excessive or undesirable levels of force and trauma are required during the process of removing the device when it is no longer needed or needs to be replaced.

### SUMMARY OF THE PRESENT INVENTION

The present invention provides a medical device that is implantable within a patient for long-term catheterization procedures. The device of the present invention includes an implantable catheterization port that is composed of a body portion, an interior port and an exterior port. The exterior port is preferably orthogonal to the interior port, such that fluids entering the body normal to the skin are then redirected along the contours of the patient's body where the interior port is connected to a catheter. The body portion is shaped to maximize comfort and ease of installation, thus a relatively flat and low-profile geometry is most preferable for a variety of applications.

The device of the present invention is composed of discrete components that are adapted for assembly at least in part in response to the unique anatomical characteristics of the patient. In particular, the body portion of the present invention may be contoured or shaped for specific applications or locations on the patient's body. Likewise, the body portion of present invention may be manufactured and distributed in various sizes and shapes such that the clinician has several options as to the overall footprint that the device will have within the patient. The interior and exterior ports are connectable to the body portion through threading or other mechanical means known in the art. As with most medical devices, the interior and exterior ports and well as the body portion are crafted from a durable and sterilizable material, such as a polymer material, stainless steel or titanium. Other suitable materials, including bio-absorbable materials, are discussed more fully below.

In one embodiment, the subcutaneous portion of the port incorporates an assembly of different tissue in-growth skirt materials that lay in concentric fashion on the top surface of the body portion. At least one of the skirts is composed of a significantly readily bio-absorbable material, while a second skirt is composed of a fabric or mesh that is significantly less bio-absorbable, and is preferably non-bio-absorbable, durable and sterile. When the device is initially placed, the skirt assembly provides a large surface area to promote tissue in-growth and securely anchor the device in position as the healing process is initiated. As the healing process progresses, the non-bio-absorbable portion of the skirt assembly gains mechanical integrity as the newly formed tissue matures, while the bio-absorbable portion of the skirt gradually lose its mechanical integrity as the material is absorbed. In this way the skirt provides an optimum level of security and stability during all phases of the healing process. By selecting one of many bio-absorbable materials for crafting the first skirt, users of the present invention can select an absorption period ranging from weeks to years, depending upon the type of procedure at hand.

These and other features and advantages of the device of the present invention are described in greater detail below with reference to the following figures.

### BRIEF DESCRIPTION OF THE DRAWINGS

FIG. 1 is a perspective view of an implantable medical device according to the present invention.

FIG. 2 is a cross-sectional view of the implantable medical device shown in FIG. 1.

FIG. 3 is an exploded perspective view of the implantable medical device shown in FIG. 1.

FIG. 4 is a plan view of the implantable medical device shown in FIG. 1.

FIG. 5 is a sectional view of the implantable medical device of the present invention shown implanted within a patient's body.

FIG. 6 is an exploded sectional view of the implantable medical device of the present invention shown implanted within a patient's body.

### DETAILED DESCRIPTION OF THE PREFERRED EMBODIMENTS

The present invention includes an implantable medical device 10 that is well adapted for providing long-term access to the inner physiology of a patient. In particular, the present invention is readily usable as a tissue ingrowth cuff or similar device for use in a patient requiring various kinds of catheterization or dialysis procedures. As the present invention includes bio-absorbable components, promotes a large degree of tissue ingrowth during the initial phases of healing, but still enables the device to be easily removed after the healing process is completed.

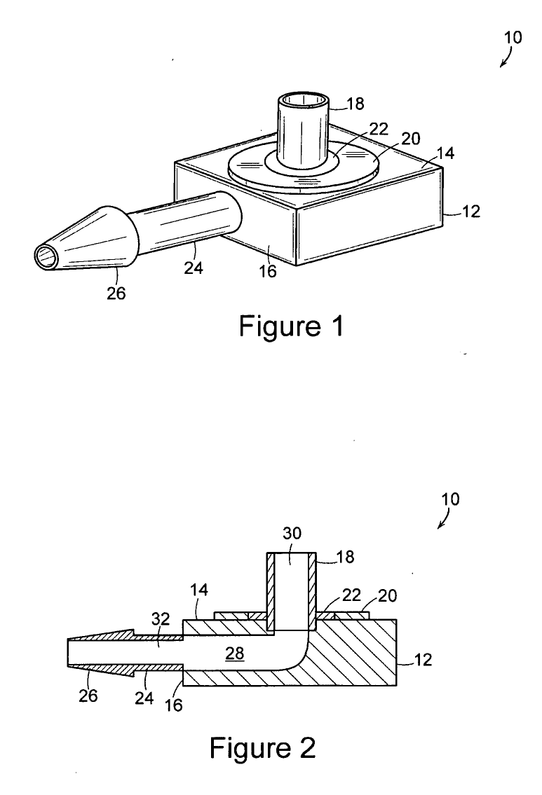

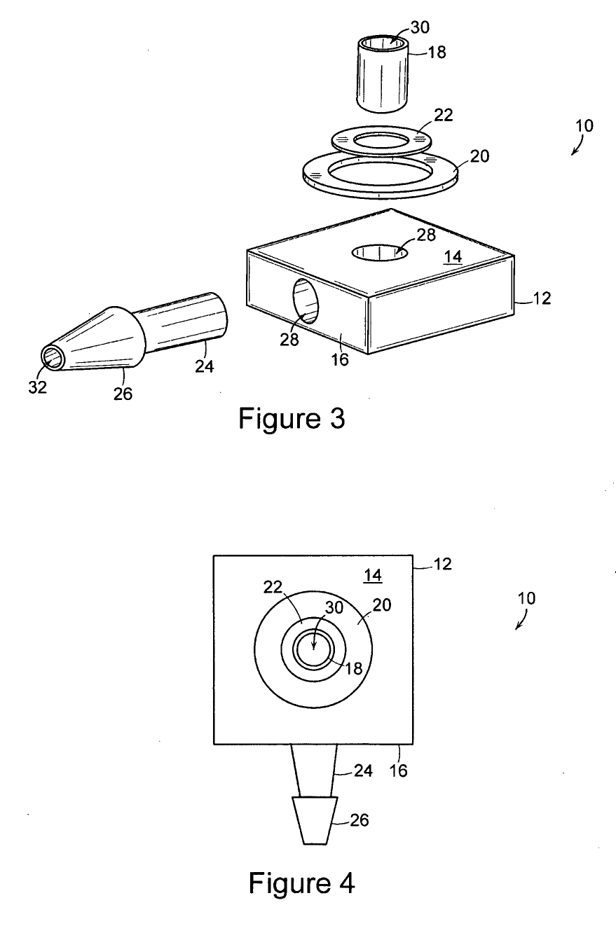

FIGS. 1 through 4 are various views of the device 10 of the present invention in one of its preferred embodiments. The device includes a body portion 12 that defines a first surface 14 and a second surface 16. In preferred embodiments, the first surface 14 and the second surface 16 are mutually orthogonal, and more preferably, the first surface 14 and the second surface 16 are arranged such that lines normal to each respective surface are mutually orthogonal. It should be understood that although the body portion 12 shown in FIGS. 1-4 defines planar surfaces, equally suitable design choices are readily available to those skilled in the art provided that the body portion 12 can contain the necessary features described below.

The body portion 12 is further adapted to receive an exterior port 18 that defines an exterior lumen 30 for transporting fluids through the exterior port 18. The exterior port 18 is preferably cylindrical in shape and may incorporate a bio-absorbable cuff material (not shown) at its junction with the body portion 12. The exterior lumen 30 preferably defines a cylindrical surface through the interior of the exterior port 18. Although a single lumen-type exterior port 18 is illustrated throughout, alternative designs including multiple lumens are readily usable according to the present invention. The exterior port 18 is selectively attachable to the body portion 12 such that the exterior port 18 may be fitted and removed by a physician. The exterior port 18 may be threaded or snapped into the body portion 12, and any number of alternative mechanical means may be used for selectively attaching the respective components consistent with the state of the art.

A first skirt 20 and a second skirt 22 disposed on the first surface 14 of the body portion 12 bound the exterior port 18. The first skirt 20 is preferably comprised of a bio-absorbable material, as discussed in further detail below. The second skirt 22 is preferably comprised of a non-bio-absorbable material, and more preferably is comprised of a durable material of the type typically used in medical device implants. Each of the first skirt 20 and the second skirt 22 preferably define an interior space adapted for receiving and surrounding the exterior port 18. More preferably, the second skirt 22 is arranged within the first skirt 20 and in closer proximity to the exterior port 18.

Accordingly, the second skirt 22 defines a shape that at its center is accepting of the exterior port 18 and at its periphery is acceptable to the center of the first skirt 20. As shown in the Figures, the first skirt 20 and the second skirt 22 are preferably annular or disk-like elements that are arranged concentrically about the exterior port 18. While this particular configuration is shown, it is understood that design and engineering decisions might otherwise dictate the preferred shape of the respective elements for particular purposes.

The body portion 12 is further adapted to receive an interior port 24 that defines an interior lumen 32 for transporting fluids through the interior port 24. The interior port 24 is preferably cylindrical in shape, and the interior lumen 32 preferably defines a cylindrical surface through the interior of the interior port 32. Although a single lumen-type interior port 24 is illustrated throughout, alternative designs including multiple lumens are readily usable according to the present invention.

The interior port 24 is selectively attachable to the body portion 12 such that the interior port 32 may be fitted and removed by a physician. The interior port 24 preferably defines a tip 26 or barb that is suitable for receiving a catheterization tube for delivering fluids into and out of the patient's system. The interior port 24 may be threaded or snapped into the body portion 12, and any number of alternative mechanical means may be used for selectively attaching the respective components consistent with the state of the art.

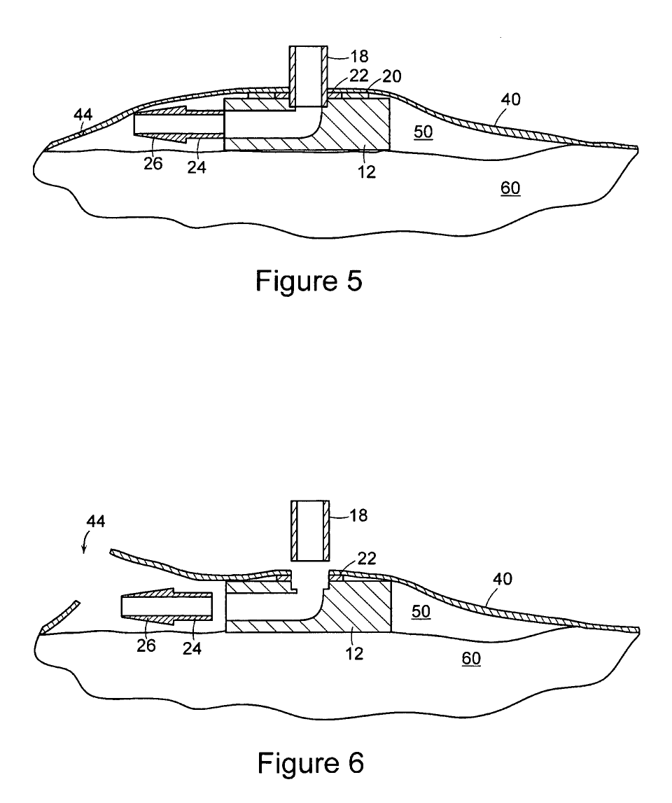

In use, the device 10 is disposed inside a patient underneath his or her skin 40 and above other tissue 60, as shown in FIGS. 5 and 6. Generally, the body portion 12 of the device will be placed under the skin 40 through an incision 44 made with a scalpel or other surgical instrument, followed by blunt dissection of the skin 40 from the tissue 60, thereby defining a pocket 50. A port or opening is generally made in the skin 40 with a scalpel or other surgical instrument for receiving the exterior port 18, while the interior port 24 is specially adapted for receiving a catheterization tube as described above.

In FIG. 5, the device 10 is shown as newly installed within the patient, such that both the first skirt 20 and the second skirt 22 are in contact with both the body portion 12 and the patient's skin 40. Over time, the first skirt 20 will begin to degrade and be absorbed by the patient's body, as it is preferably comprised of a bio-absorbable material. Thus, as time elapses, the device 10 will appear as shown in FIG. 6, with only the second skirt 22 disposed on the first surface 14.

As the second skirt 22 becomes integrated with the patient's skin 40, the device 10 becomes more permanently secured within the patient. Once the bio-absorbable first skirt 20 is fully degraded and absorbed by the patient's body, the device 10 of the present invention can be more easily removed from the patient's body through normal surgical means.

As previously noted, the first skirt 20 is preferably comprised of a bio-absorbable material such that it secures the device 10 within the patient's body and then gradually mechanically degrades prior to removal of the device. A host of bio-absorbable materials are known in the art of medical devices. Preferable materials for the present invention include polymers such as polyglycolide, polylactide, l-lactide, poly(dl-lactide), polycolactide, poly(ε-caprolactone), polydiaxanone, polyglyconate or poly(lactide-co-glycolide) (DLPLG). Different formulas of the DLPLG may be used, including 85/15, 75125, 65/35 and 50/50 wherein the various ratios are indicative of the mixtures of glycolide and dl-lactide, respectively. The higher the proportional ratio of glycolide to dl-lactide, the greater the period of degradation of the DLPLG polymer.

Each of these polymers has unique mechanical and thermal properties, as well as variable degradation intervals. Accordingly, the type of polymer selected for the first skirt 20 is at least partially dependent upon the mechanical requirements of the application as well as the time period for which the device 10 must be placed within a patient. For example, polyglycolide will remain in the human body for approximately six to twelve months, while polylactide will remain for more than twenty-four months. Similarly, poly(dl-lactide) will remain in tact within the body for approximately twelve to sixteen months, while polycolactide will last over two years in a patient. Poly(ε-caprolactone), polydiaxanone, polyglyconate and the various forms of poly(lactide-co-glycolide) all have a duration of between one and twelve months. Thus, the type of material selected for the first skirt 20 should be selected at least in part for its durability and interval of degradation. Longer-term implantations would require materials with a longer half-life, while the opposite is true for more short-term applications of the device 10.

The second skirt 22 is preferably comprised of a non-bio-absorbable material, such as for example polyester fabric, woven polyurethane or titanium mesh. Similarly, the body portion 12 and the respective ports are preferably comprised of durable, non-bio-absorbable materials such as polyurethane, polysulfone, polycarbonate, silicone, titanium or stainless steel. Any combination of materials is also suitable for the foregoing components, such that they will be lightweight, durable and easy to assemble and maintain once inside the patient's body.

Although the second skirt 22, the body portion 12 and the respective ports are preferably composed of those materials described above, it is also possible and in some cases preferable to manufacture these components out of bio-absorbable materials. As there are varying rates of degradation, as noted above, it is possible to select materials that will have degradation periods on the order of years as opposed to months. Thus the entirety of the present device may be composed of bio-absorbable materials, subject to the application and the interval that the device 10 is expected to be in use. For example, the first skirt 20 may be composed of polyglycolide, while the remaining components may be composed of polycolactide, which will render the remaining components effectively permanent relative to the first skirt 20 and the expected duration of the application. Accordingly, the present invention can be constructed in a number of fashions depending upon the intended use of the device 10.

The device 10 of the present invention is assembled or manufactured such that the first skirt 20 and the second skirt 22 can be selectively attached to the body portion 12 by mechanical or other means, including adhesives. The exterior port 18 and the interior port 24 are preferably snap-fit or threaded into the body portion 12, such that removal is possible at any time during the use of the device 10. It is worth noting that adhesive will affect the functionality of any bio-absorbable materials, so in preferred embodiments, the first skirt 20 has little or no contact with any adhesives.

As described herein, the present invention includes a device 10 that is readily usable as a tissue ingrowth cuff or similar device for permitting vascular access to a patient for various kinds of catheterization or dialysis procedures. The present invention includes a bio-absorbable skirt about an exterior port, thus making it is easier to maintain during the therapeutic period and easier to remove from the patient following thereafter. As the duration of the catheterization procedure will vary depending upon the type of procedure, the present invention is adapted for filting with a number of bio-absorbable skirts, each of which has a unique range over which it will be absorbed into the patient's body. Thus for long-term applications, such as dialysis, a more resilient bio-absorbable skirt may be used. More short-term applications may utilize a bio-absorbable skirt that will degrade within a number of weeks or months. Moreover, as the present invention is modular by nature, it is possible to assemble the device on site, thus giving the physician or technician the option of choosing between a range of bio-absorbable skirts during final assembly and installation of the device.

Although the present invention has been described above with reference to preferred embodiments and materials, it should be understood that the scope thereof it not so limited. Numerous adaptations to the preceding description can be readily devised by those skilled in the art without departing from the spirit and scope of the present invention as defined in the following claims.

I claim:

1. An implantable medical device comprising:
a body portion defining a first surface and a body lumen, the body lumen defining an exterior portion and an interior portion substantially orthogonal to the exterior portion;
an exterior port defining an exterior lumen, the exterior port selectively joinable to the body portion normal to the first surface such that the exterior lumen is substantially orthogonal to the interior portion of the body lumen; and
a first skirt comprised of a bio-absorbable material disposed about the exterior port such that the skirt is disposed on the first surface of the body portion.

2. The device of claim 1 further comprising an interior port defining an interior lumen, the interior port selectively joinable to the body portion substantially orthogonal to the exterior port.

3. The device of claim 1 further comprising a fitting disposed about the exterior port for selectively attaching a catheterization device thereto.

4. The device of claim 1 wherein the first skirt is selectively attachable to the body portion.

5. The device of claim 1 wherein the first skirt is selectively attachable to the exterior port.

6. The device of claim 1 further comprising a second skirt disposed about the exterior port such that the second skirt is disposed on the first surface of the body portion.

7. The device of claim 6 wherein the second skirt is disposed anterior to the first skirt relative to the exterior port.

8. The device of claim 1 wherein the bio-absorbable material is a polymer material.

9. The device of claim 1 wherein the bio-absorbable material is selected from the group consisting of polyglycolide, polylactide, l-lactide, poly(dl-lactide), polycolactide, poly(ε-caprolactone), polydiaxanone or poly(lactide-co-glycolide).

10. The device of claim 1 wherein the bio-absorbable material is polyglycolide, polylactide, l-lactide, poly(dl-lactide), polycolactide, poly(ε-caprolactone), polydiaxanone, polyglyconate, or (lactide-co-glycolide).

11. The device of claim 1 wherein the bio-absorbable material is polyglycolide.

12. The device of claim 1 wherein the bio-absorbable material is polylactide.

13. The device of claim 1 wherein the bio-absorbable material is l-lactide.

14. The device of claim 1 wherein the bio-absorbable material is poly(dl-lactide).

15. The device of claim 1 wherein the bio-absorbable material is polycolactide.

16. The device of claim 1 wherein the bio-absorbable material is poly(ε-caprolactone).

17. The device of claim 1 wherein the bio-absorbable material is polydiaxanone.

18. The device of claim 1 wherein the bio-absorbable material is polyglyconate.

19. The device of claim 1 wherein the bio-absorbable material is poly(lactide-co-glycolide).

20. The device of claim 1 wherein the body portion is comprised of a material selected from the group consisting of polyurethane, polysulfone, polycarbonate, silicone, titanium or stainless steel.

21. The device of claim 1 wherein the body portion is comprised of polyurethane.

22. The device of claim 1 wherein the body portion is comprised of polysulfone.

23. The device of claim 1 wherein the body portion is comprised of polycarbonate.

24. The device of claim 1 wherein the body portion is comprised of silicone.

25. The device of claim 1 wherein the body portion is comprised of titanium.

26. The device of claim 1 wherein the body portion is comprised of stainless steel.

27. The device of claim 6 wherein the second skirt is comprised of a material selected from the group consisting of polyester fabric, woven polyurethane or titanium mesh.

28. The device of claim 6 wherein the second skirt is comprised of polyester fabric.

29. The device of claim 6 wherein the second skirt is comprised of woven polyurethane.

30. The device of claim 6 wherein the second skirt is comprised of titanium mesh.

31. The device of claim 1 wherein the body portion and the exterior port are comprised of a bio-absorbable material.

32. The device of claim 2 wherein the interior port is comprised of a bio-absorbable material.

33. The device of claim 6 wherein the second skirt is comprised of a bio-absorbable material.
